(19) United States
(12) Patent Application Publication
(10) Pub. No.: US 2026/0165676 A1
(43) Pub. Date: Jun. 18, 2026

(54) ARRAY-TYPE FOCUSED ULTRASOUND TRANSDUCER AND MEDICAL ULTRASOUND DEVICE

(71) Applicant: ECO DM LAB CO., LTD., Cheongju-si, Chungcheongbuk-do (KR)

(72) Inventors: Man Soon YOON, Cheongju-si, Chungcheongbuk-do (KR); Young Min PARK, Cheongju-si, Chungcheongbuk-do (KR); Jae Hyouk CHOI, Cheongju-si, Chungcheongbuk-do (KR)

(21) Appl. No.: 18/711,377

(22) PCT Filed: Feb. 19, 2024
(86) PCT No.: PCT/KR2024/095413

§ 371 Date: May 17, 2024

(30) Foreign Application Priority Data
Feb. 20, 2023 (KR) 10-2023-0022419

(51) Int. Cl. A61B 8/12; A61B 8/00; A61B 8/08

(52) U.S. Cl. CPC A61B 8/12; A61B 8/0825; A61B 8/4483

(57) ABSTRACT

A dome-type focusing-type ultrasound transducer capable of effectively focusing focused ultrasound waves and thus having high efficiency, and a medical ultrasound device including the same are described. An example of the focused ultrasound transducer may include a dome-shaped support and a plurality of ultrasound transducer cells coupled to the dome-shaped support, the ultrasound transducer cell includes a metal vibrating body whose first surface is disposed toward an inner side thereof, which is a concave direction of the dome-shaped support, and a piezoelectric ceramic element disposed on a second surface, which is a surface opposite to the first surface of the metal vibrating body, and the first surface of the metal vibrating body has a predetermined radius of curvature so as to be concave toward the inner side of the dome-shaped support.

### TECHNICAL FIELD

The present invention relates to an array-type focused ultrasound transducer in which a plurality of transducer cells are disposed on a dome-shaped support and a medical ultrasound device including the same, and relates to an array-type focused ultrasound transducer in which a metal vibrating body having a predetermined radius of curvature is disposed on the front of the plurality of transducer cells.

### BACKGROUND ART

Ultrasound technology is widely used in the medical, industrial, and environmental processing fields, and recently, as interest in beauty has increased, a variety of devices using ultrasound have been introduced in order to prevent skin aging, improve wrinkles, or maintain skin elasticity. In addition, it is also used to in a method of treating depression, etc. by stimulating cranial nerves using a low-intensity, low-frequency (100 KHz to 1 MHz) ultrasound focusing device to allow drugs used to treat brain diseases to spread smoothly, or by stimulating the cranial nerves using low-power ultrasound.

A piezoelectric element for focusing ultrasound waves used for this purpose selects a location to focus ultrasound waves by tomography of the brain, and then selects a mounting location of an ultrasound piezoelectric element with a predetermined focal length in order to allow ultrasound waves to be transmitted to the selected location, and periodically oscillates ultrasound waves to stimulate or activate the nerves at a procedure site.

FIG. 1 shows an ultrasound focusing device for treating brain diseases, and FIG. 2 is a schematic diagram illustrating treatment by focusing ultrasound waves from a dome-type ultrasound focusing transducer so as to allow the ultrasound waves to be focused on a brain disease area of a patient.

FIG. 3 shows piezoelectric elements arranged in this dome-shaped ultrasound transducer. By arranging so many piezoelectric elements in a dome shape, ultrasound waves are guided to be focused inside the dome. For the piezoelectric elements, a dome-shaped ultrasound focusing transducer with a large diameter is required to fit the size of the brain, as shown in FIG. 2. In order to minimize scattering, the smaller the diameter of the flat piezoelectric ceramic, the smaller the aberration can be. Therefore, at least 12,000 piezoelectric elements with a diameter of 2 to 3 mm are required, which poses a great difficulty in manufacturing it.

FIGS. 4 and 5 show a conventional dome-shaped ultrasound transducer array. It shows a dome-shaped transducer for ultrasound treatment composed of a support 24 having a constant radius of curvature surrounding the brain and a sub-cell 22 in which piezoelectric elements radiating ultrasound waves toward the brain are arranged by being attached to the support 24. A plurality of piezoelectric elements 29 are disposed in the sub-cell 22 and ultrasound waves are generated from a surface 28 thereof toward the inside of the dome.

However, these piezoelectric elements 29 are of a flat type, and when driving this dome-shaped ultrasound transducer, the ultrasound waveforms generated from each of the piezoelectric elements arranged in an array are dispersed, and thus it is difficult to focus so that a problem that the focus is dispersed (FIG. 6) or focusing is made by being shifted away from the desired area may easily occur.

To solve this problem, U.S. Pat. No. 6,613,005 describes a dome-type ultrasound focusing transducer that can finely adjust the unit cell. As shown in FIG. 7, it has a structure that receives focus information and controls focus dispersion by installing a two-dimensional gimbal device 26 and an actuator 18 capable of moving the sub-cell 22 to provide degrees of freedom in two arrow directions.

In the dome-shaped ultrasound focusing transducer manufactured in this way, a circuit for driving the gimbal device 26 and actuator 18 is connected to a circuit that drives the unit cell to radiate ultrasound waves toward the inside of the dome and control is made so that the focus is formed on the desired treatment area.

However, in such an ultrasound transducer for treating brain diseases, the piezoelectric elements 28 constituting the unit cell are flat, and thus the radiation of ultrasound waves is radiated perpendicular to the plane as shown in FIG. 8, thereby causing dispersion of focus. In order to prevent dispersion of the focus, the gimbal device 26 and the actuator 18 should be controlled separately, and thus the program is complicated and the procedure time increases. In addition, since the resonance frequencies of respective piezoelectric elements 28 should match, a manufacturing process thereof is complicated, and since the actuator and the gimbal device for controlling each sub-cell are required for each sub-cell, it has a structure that inevitably increases manufacturing costs. In addition, since the case filled with fluid requires waterproofing, a precise actuator waterproof structure used is also required. In addition, the device is complicated, and if a single unit cell is damaged, the sub-cell 22 containing three piezoelectric elements 28 should be discarded, and thus maintenance costs are increased.

### DISCLOSURE OF THE INVENTION

### Technical Problem

An object of the present invention is to provide a dome-type focusing-type ultrasound transducer capable of effectively focusing focused ultrasound waves and thus having high efficiency, and a medical ultrasound device including the same.

In addition, another object thereof is to provide a dome-type focusing-type ultrasound transducer that can be manufactured at a low manufacturing cost due to its simple structure and manufacturing process and has low maintenance costs, and a medical ultrasound device including the same.

### Technical Solution

In order to achieve the objects described above, a focused ultrasound transducer according to the present invention includes a dome-shaped support and a plurality of ultrasound transducer cells coupled to the dome-shaped support, the ultrasound transducer cell includes a metal vibrating body whose first surface is disposed toward an inner side thereof, which is a concave direction of the dome-shaped support, and a piezoelectric ceramic element disposed on a second surface, which is a surface opposite to the first surface of the metal vibrating body, and the first surface of the metal vibrating body has a predetermined radius of curvature so as to be concave toward the inner side of the dome-shaped support.

In addition, in the focused ultrasound transducer according to an embodiment of the present invention, the radius of curvature of the first surface of the metal vibrating body may be the same as a radius of curvature of the inner side of the dome-shaped support.

In addition, in the focused ultrasound transducer according to the embodiment of the present invention, the radius of curvature of the first surface of the metal vibrating body may be smaller than the radius of curvature of the inner side of the dome-shaped support.

In addition, in the focused ultrasound transducer according to the embodiment of the present invention, a seating groove may be formed on the second surface of the metal vibrating body and the piezoelectric ceramic element is seated in the seating groove.

In addition, in the focused ultrasound transducer according to the embodiment of the present invention, a plurality of holes may be formed in the dome-shaped support, and the plurality of ultrasound transducer cells may be respectively inserted into the plurality of holes.

In addition, in the focused ultrasound transducer according to the embodiment of the present invention, an inner wall of the hole and an outer periphery of the metal vibrating body may be screw-coupled to each other.

In addition, in the focused ultrasound transducer according to the embodiment of the present invention, the hole has a catching step inside thereof, and a catching portion that restricts an inward movement of the dome-shaped support by the catching step is formed on the metal vibrating body of the ultrasound transducer cell.

In addition, in a focused ultrasound transducer array according to an embodiment of the present invention, a first wall extending from an edge of the second surface may be formed, and the catching portion may be formed on an outer peripheral part of the first wall.

In addition, in the focused ultrasound transducer according to the embodiment of the present invention, a vibrating body fixing portion that fixes the ultrasound transducer cell by inserting the transducer cell into the hole from an outside of the dome-shaped support after being inserted into the hole to make the catching portion of the metal vibrating body and the catching step of the hole come into close contact with each other may be included.

In addition, in the focused ultrasound transducer according to the embodiment of the present invention, the vibrating body fixing portion may have a bolt structure that has a through-hole and is screw-coupled to the inner wall of the hole.

In addition, in the focused ultrasound transducer according to the embodiment of the present invention, a sealing member may be disposed on a surface where the catching portion comes into contact with the catching step of the hole.

In addition, in the focused ultrasound transducer according to the embodiment of the present invention, the metal vibrating body may be made of stainless steel.

In addition, in the focused ultrasound transducer according to the embodiment of the present invention, the dome-shaped support may be made of an engineering plastic material.

Meanwhile, a medical ultrasound device including the focused ultrasound transducer according to the present invention may be provided.

### Advantageous Effects

Through the dome-shaped focused ultrasound transducer according to the present invention, effective focusing of focused ultrasound is made, thereby capable of increasing the effectiveness of ultrasound treatment or diagnosis and reducing the procedure time.

In addition, the manufacturing costs and maintenance costs of the dome-type focused ultrasound transducer according to the present invention and the medical device including the same can be reduced, thereby reducing the costs required for ultrasound treatment or diagnosis.

### BRIEF DESCRIPTION OF THE DRAWINGS

FIG. 1 shows an example of an ultrasound medical device for treating a brain disease.

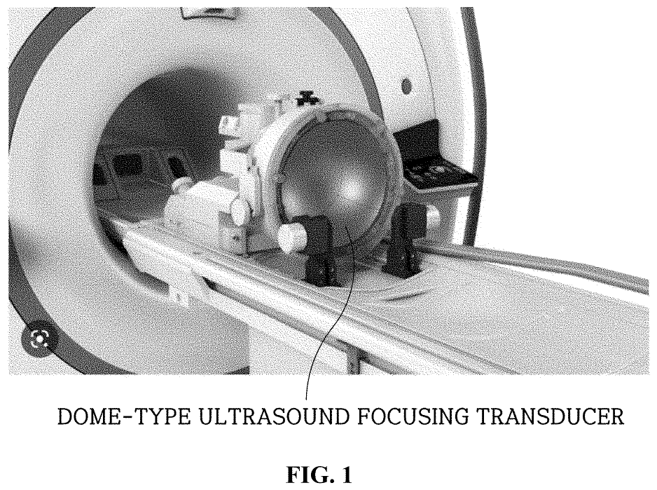

FIG. 2 is a schematic diagram schematically showing a principle of treating the brain disease using an array-type ultrasound focusing device.

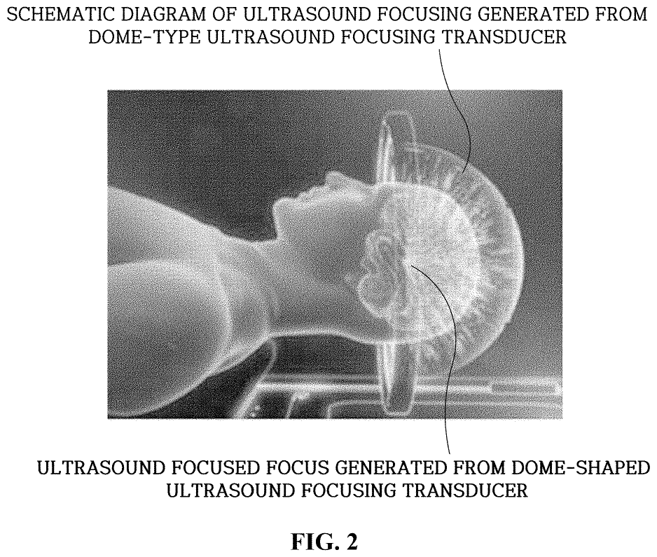

FIG. 3 is a schematic diagram showing a piezoelectric element array arranged in a dome-type ultrasound focusing transducer.

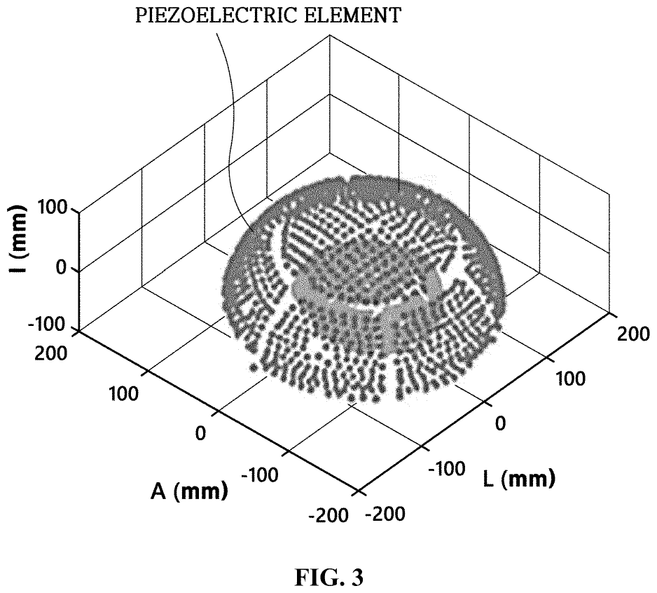

FIG. 4 is a schematic diagram showing a conventional dome-type ultrasound focusing transducer.

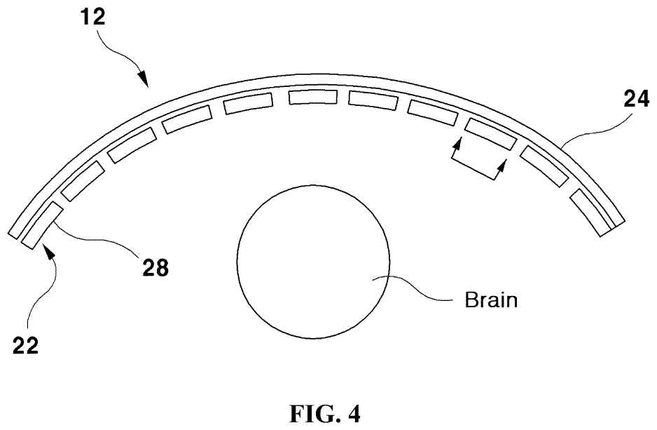

FIG. 5 is a schematic diagram showing a structure of a unit cell of the conventional dome-type ultrasound focusing transducer.

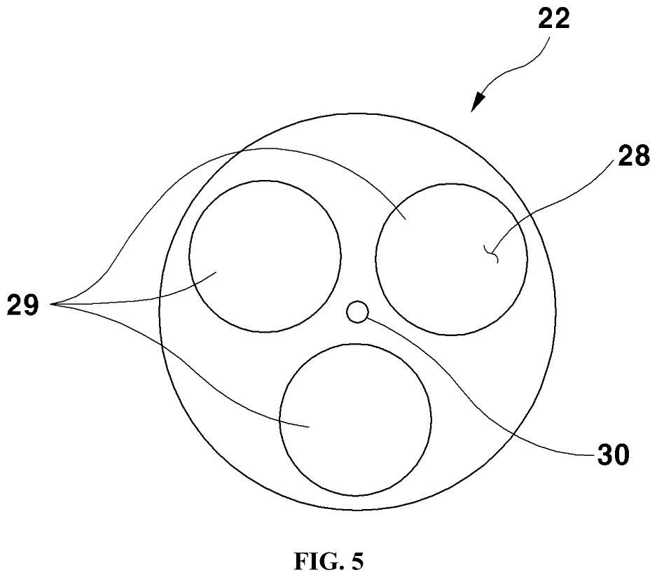

FIG. 6 is a schematic diagram illustrating dispersion of focus that occurs when driving the conventional dome-type ultrasound focusing device.

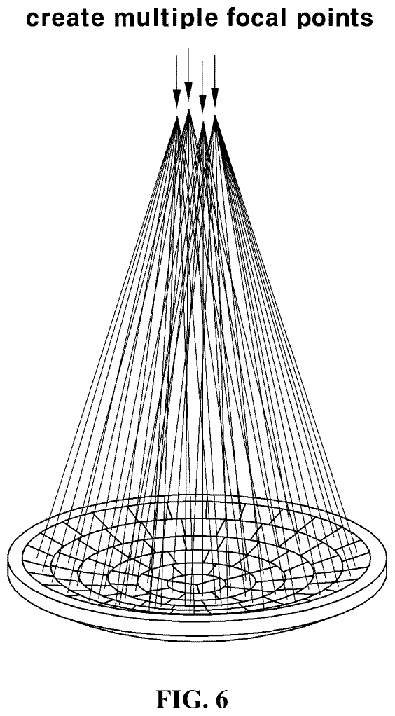

FIG. 7 is a schematic diagram showing an actuator that drives the unit cell of the conventional dome-type ultrasound focusing transducer.

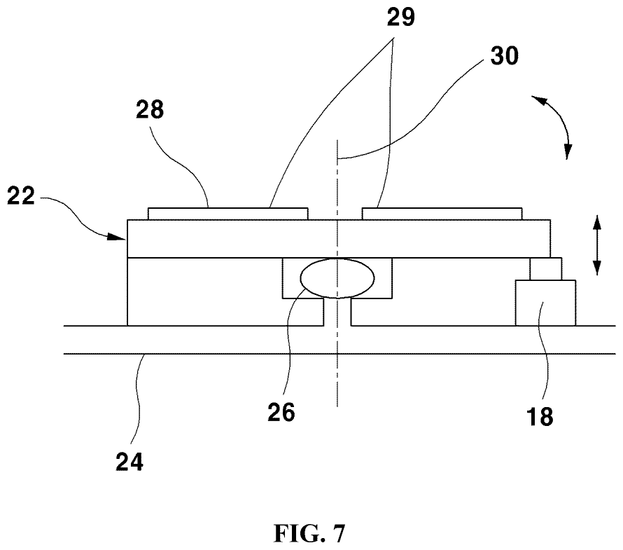

FIG. 8 is a schematic diagram illustrating ultrasound focusing when the conventional dome-type ultrasound focusing transducer is driven.

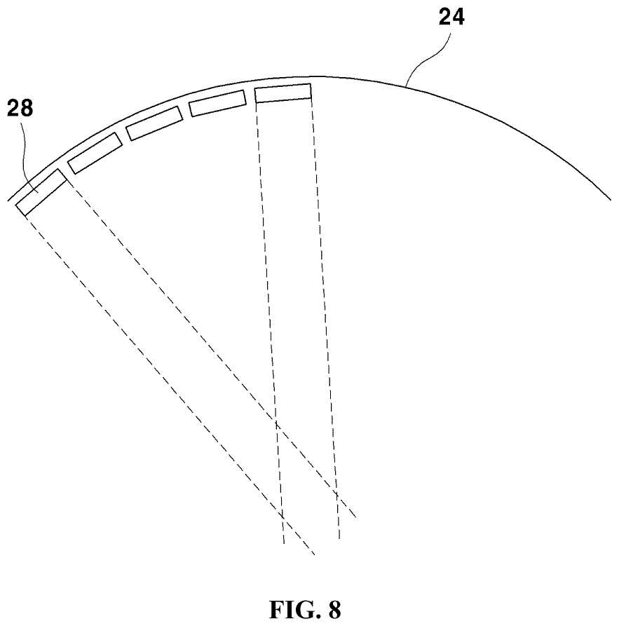

FIG. 9 is a schematic diagram illustrating a unit transducer cell according to an embodiment of the present invention.

FIG. 10 is a schematic diagram illustrating ultrasound wave focusing by a dome-shaped arrangement of unit transducer cells according to the embodiment of the present invention.

FIG. 11 is a schematic diagram illustrating amplification of displacement through a metal vibrating body in the transducer cell according to an embodiment of the present invention.

FIG. 12 is a top view and a perspective view for describing a dome-shaped support in the ultrasound focusing transducer according to an embodiment of the present invention.

FIG. 13 is a diagram illustrating a metal vibrating body whose ultrasound radiation surface has a radius of curvature in the ultrasound focusing transducer according to an embodiment of the present invention.

FIG. 14 is a diagram illustrating a hollow-type bolt structure for fixing individual transducer cells in the ultrasound focusing transducer according to the present invention.

FIG. 15 is a diagram showing a state in which the transducer cell is fixed to a support through a vibrating body fixing portion in the ultrasound focusing transducer according to the present invention.

FIG. 16 is a diagram showing an ultrasound focusing transducer completed according to the present invention.

### MODE FOR CARRYING OUT THE INVENTION

Hereinafter, embodiments of the present invention will be described in detail with reference to the accompanying drawings so that those of ordinary skill in the art to which the present application belongs can easily implement them. However, the present application may be implemented in various different forms and is not limited to the embodiments described herein.

In addition, in order to clearly describe the present application in the drawings, parts unrelated to the description have been omitted, and similar reference numerals have been assigned to similar parts throughout the specification.

Throughout the specification of the present application, when a part is said to be “connected” to another part, this includes not only cases where it is “directly connected,” but also cases where it is “electrically connected” with another element in between.

Throughout the specification of the present application, when a member is said to be located “on”, “on an upper part”, “at the top”, “below”, “at a lower part”, or “at the bottom” of another member, this includes not only cases where the member is in contact with another member, but also cases where another member exists between two members.

Throughout the specification of the present application, when a part is said to “include” a certain constituent element, this means that it can further include other constituent elements rather than excluding other constituent elements, unless specifically stated to the contrary.

As used in this specification, the terms “approximately”, “substantially”, etc. are used to mean at or close to that numerical value when manufacturing and material tolerances unique to the meaning mentioned are given, and are used to prevent unscrupulous infringers from unfairly using the disclosed contents in which accurate or absolute numerical values are mentioned in order to aid the understanding of the present invention. In addition, throughout the specification of the present application, “a step of doing ˜” or “a step of ˜” does not mean “a step for ˜”.

Throughout the specification of the present application, the term “combination thereof” means a mixture or combination of one or more selected from a group consisting of the constituent elements described in the the expression of the Makushi format, and means including one or more selected from the group consisting of the constituent elements.

Throughout the specification of the present application, the description of “A and/or B” means “A or B, or A and B.”

In addition, unless otherwise defined, all technical and scientific terms have the same meaning as commonly understood by one of ordinary skill in the art to which the present invention belongs. The terms used in the description herein are intended only to effectively describe particular embodiment and is not intended to limit the present invention.

The dome-shaped focused ultrasound transducer according to the present invention includes a dome-shaped support and a plurality of ultrasound transducer cells coupled to the dome-shaped support, the ultrasound transducer cell includes a metal vibrating body whose first surface is disposed toward an inner side, which is in a concave direction of the dome-shaped support, and a piezoelectric ceramic element disposed on a second surface, which is a surface opposite to the first surface of the metal vibrating body, and the first surface of the metal vibrating body has a predetermined radius of curvature so as to be concave toward the inner side of the dome-shaped support.

A conventional dome-type focused ultrasound transducer required a complicated device in order to focus ultrasound waves generated from multiple ultrasound transducer cells. However, in the dome-type focused ultrasound transducer according to the present invention, each transducer cell includes a piezoelectric element that generates ultrasound waves and a metal vibrating body having a constant radius of curvature in front of the piezoelectric element. Through this, it is possible to effectively focus the ultrasound waves generated from each transducer cell on a desired area without the complicated device.

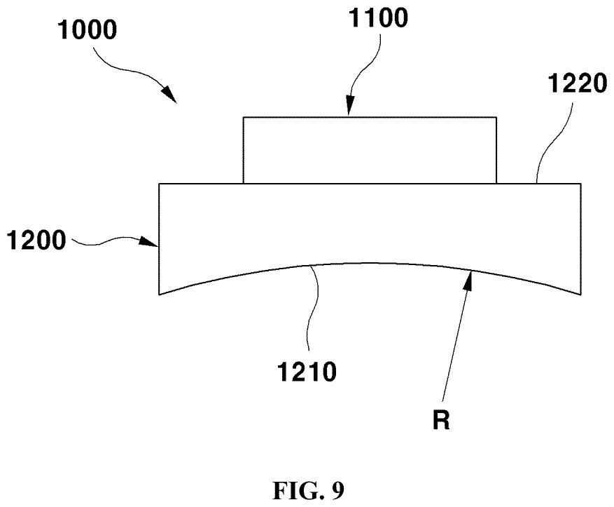

FIG. 9 is a diagram illustrating an individual transducer cell in a focused ultrasound transducer according to an embodiment of the present invention. A metal vibrating body 1200 is disposed in front of a piezoelectric ceramic element 1100 that generates ultrasound waves. A first surface 1210 of the metal vibrating body 1200 in the direction in which ultrasound waves are radiated has a predetermined radius of curvature R. As the transducer cell having the radius of curvature R is disposed on the dome-shaped support, ultrasound focusing can be effectively performed. In this case, the radius of curvature R of the first surface 1210 of the metal vibrating body 1200 and the radius of curvature of the dome-shaped support may be the same. However, they may not be the same and may be different from each other, and the focus for focusing can be appropriately changed through the change in the radius of curvature.

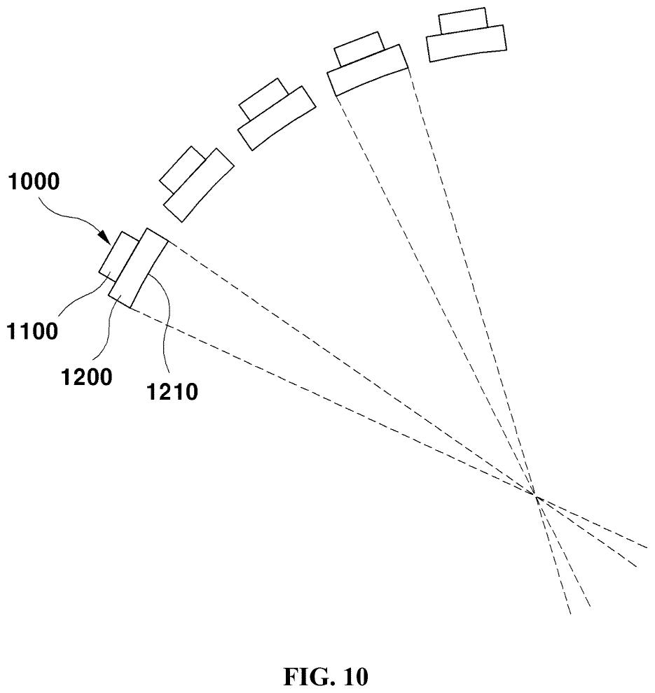

FIG. 10 illustrates a transducer cell array in which a transducer cell 1000 including the metal vibrating body 1200 according to the present invention is disposed on a support and the ultrasound focusing according to the disposition. Ultrasound waves are effectively focused through the first surface 1210 of the metal vibrating body 1200 having a predetermined radius of curvature.

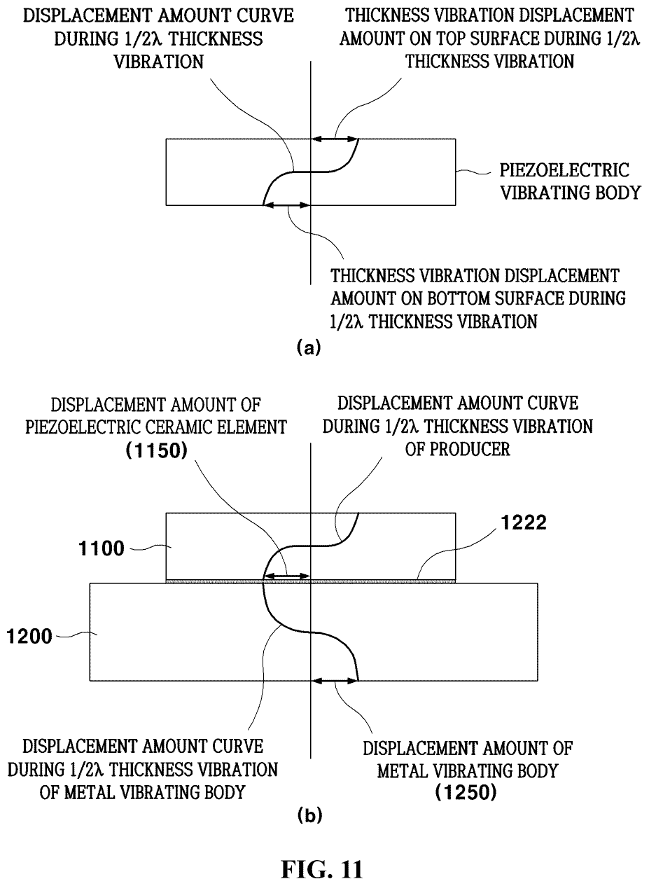

Vibration through the metal vibrating body will be described in FIG. 11. FIG. 11(a) is a diagram illustrating vibration in a piezoelectric vibrating body of a general single structure. Vibration when the piezoelectric vibrating body is obtained by coupling the piezoelectric ceramic element 1100 and the metal vibrating body 1200 as in the present invention will be described with reference to FIG. 11(b). In FIG. 11(b), since the left-right vibration of the piezoelectric ceramic element 1100 has an amplification effect through the metal vibrating body 1200, an amplitude 1150 in the piezoelectric ceramic element 1100 is amplified to an amplitude 1250 in the metal vibrating body, so that it becomes possible to generate high sound pressure at the same energy.

However, since a thickness of the metal vibrating body 1200 according to the present invention has a curvature, the thicknesses of the edge and the center thereof are different. Considering the radius of curvature and the diameter of the metal horn, it is necessary to design it so that a rate of reduction in amplification due to the thickness of the edge is the lowest. Referring to FIG. 11, as in FIG. 11(b), when the piezoelectric ceramic element 1100 and the metal vibrating body 1200 are bonded to each other through an adhesive layer 1222, in order to efficiently transmit a vibration displacement at a primary resonance mode to the metal vibrating body 1200 in a case where vibration of 600 kHz is applied, the metal vibrating body 1200 should also be vibrated in a thickness vibration mode of ½λ mode. For example, in a case where stainless steel (SUS 303 material) having a sound velocity of 5,640 m/s is used as a material of the metal vibrating body 1200, the thickness of the metal vibrating body 1200 is determined by Equation 1 below in order for the metal vibrating body 1200 to vibrate in the ½λ mode.

$${t}_{1} = \frac{1}{z} \times \frac{v}{{f}_{r 1}} \qquad [ Equation 1 ]$$

Here, t₁ represents the thickness (mm) of the piezoelectric ceramic element 1100 in ½λ mode, v represents the speed of sound (m/s) in the thickness mode of the metal vibrating body 1200, and fᵣ₁ represents the primary resonance frequency.

That is, the primary resonance frequency in the ½λ mode of the piezoelectric ceramic element 1100 is 600 kHz, and the material of the metal vibrating body 1200 is SUS 303, which is stainless steel, and the speed of sound thereof is 5,640 m/s, and thus when the thickness of the metal vibrating body 1200 is 4.7 mm, the vibration of the piezoelectric ceramic element 1100 can be most efficiently transmitted through the metal vibrating body 1200. In addition, as shown in FIG. 11(b), a vibration displacement 1150 due to vibration of the piezoelectric ceramic element 1100 appears as an amplified displacement 1250 as the vibration passes through the inside of the metal vibrating body 120, so that displacement amplification can be efficiently amplified.

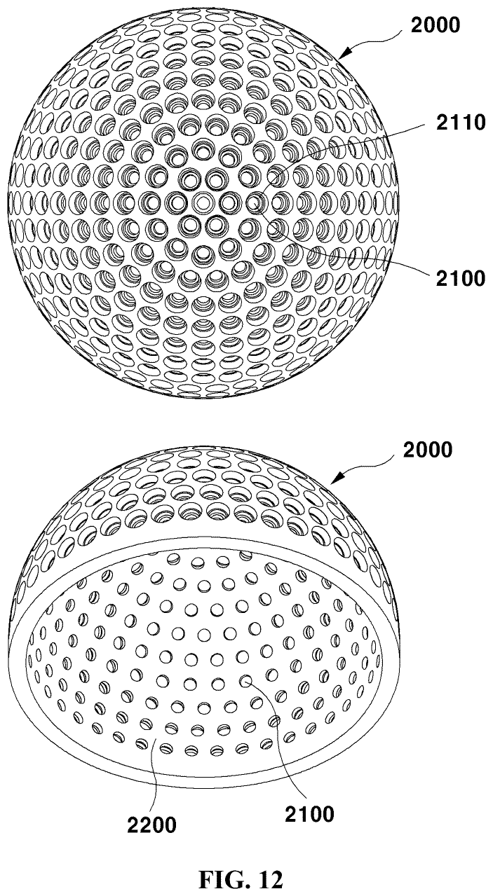

FIG. 12 shows a dome-shaped support 2000 to which the transducer cell for ultrasound generation is coupled. A hole 2100 into which a transducer cell for ultrasound generation can be coupled is formed on this support. In addition, a step 2110 on which the cell can be seated is formed inside each hole 2100. This support 2000 may be a high-strength engineering plastic such as PEEK or may be metal.

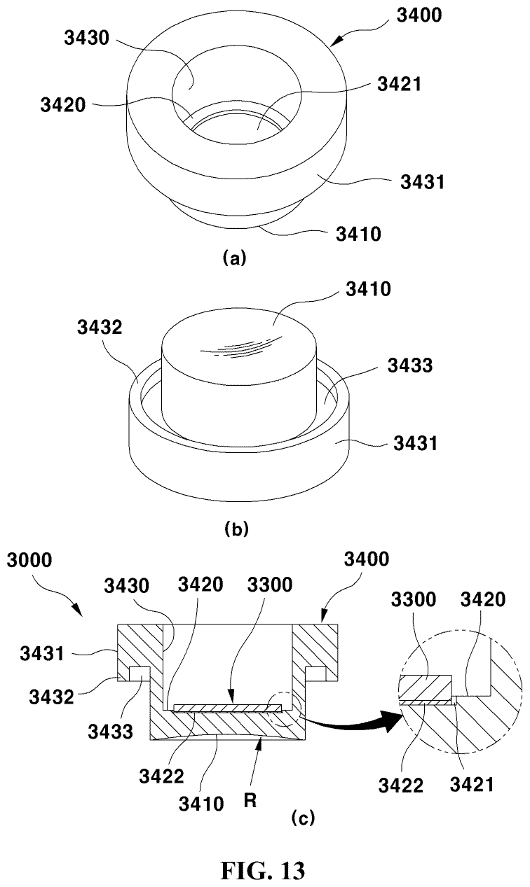

FIG. 13(a) and FIG. 13(b) are perspective views of a metal vibrating body 3400 whose ultrasound radiation surface used in the present invention has a radius of curvature. FIG. 13(c) shows a cross section of a transducer cell 3000 in which a piezoelectric ceramic element 3300 for generating ultrasound waves is attached to a seating groove 3421 formed on an upper surface 3420 of the metal vibrating body using epoxy. The surface that radiates ultrasound waves to human tissue is a lower surface 3410 of the metal vibrating body 3400 and has a predetermined radius of curvature R, and the thickness between the upper surface 3420 and the lower surface 3410 has a thickness calculated by Equation 1. In addition, an inner wall 3430 extending from the edge of the upper surface of the metal vibrating body 3400 is formed, and a catching protrusion 3432 protruding from the outer periphery of the inner wall 3430 is formed, and thus, when the transducer cell 3000 is inserted into the hole 2100 of the dome-shaped support 2000, it is caught on the step 2110 inside the hole 2100 and its movement toward the inner side of the dome-shaped support 2000 is restricted.

In addition, a groove 3433 into which a sealing member such as a rubber ring can be inserted is formed on the catching protrusion 3432 for sealing. In addition, a guide wall 3431 whose diameter is equal to the diameter of the hole 2100 of the support 2000 is formed.

Meanwhile, according to an embodiment of the present invention, a vibrating body fixing portion that fixes the ultrasound transducer cell by additionally inserting the transducer cell into the hole from an outside of the support after being inserted into the hole to make the catching protrusion formed on the the metal vibrating body and the catching step of the hole come into close contact with each other may be included.

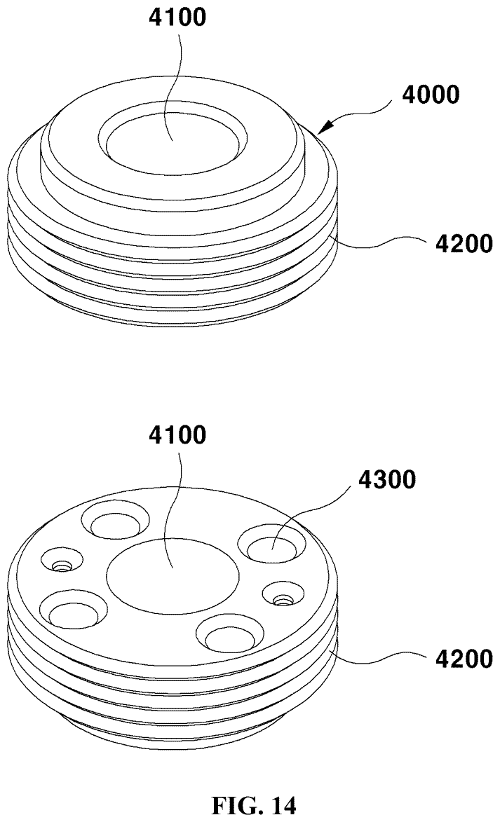

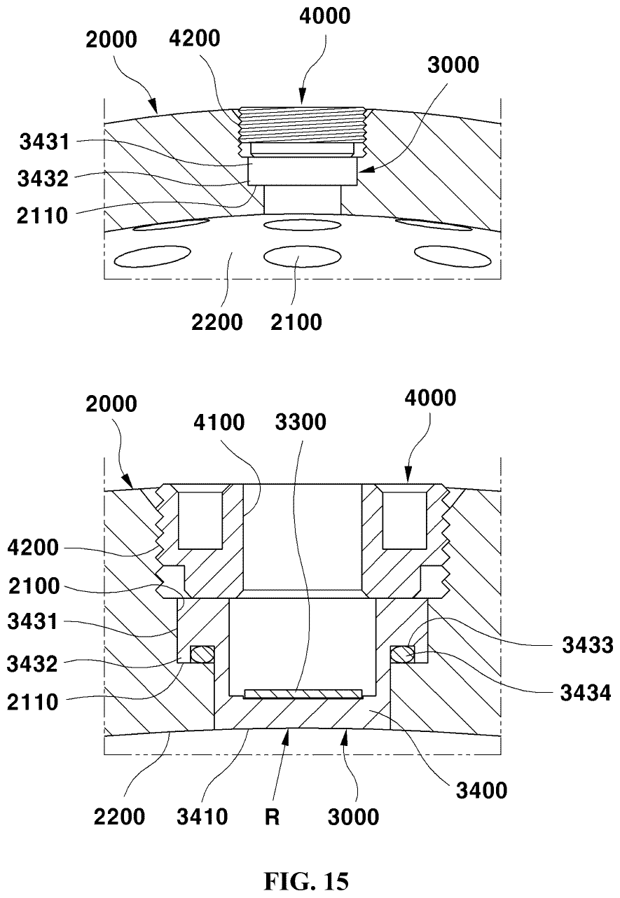

Examples of this vibrating body fixing portion and the transducer cell fixed to the dome-shaped support through it are shown in FIGS. 14 and 15.

A vibrating body fixing portion 4000 has a through-hole 4100, and a thread 4200 is formed on the outer periphery thereof for screw-coupling with the inner wall of the hole. The through-hole 4100 is for connecting a wire for applying electricity to the piezoelectric ceramic element 3300 from the outside. In addition, a fastening hole 4300 for using a tooth tool for screw-coupling with the support 2000 may be formed.

FIG. 15 illustrates a state in which the transducer cell 3000 is coupled to the support 2000 through the vibrating body fixing portion 4000. First, the transducer cell 3000 is inserted into the hole 2100 and the catching protrusion 3432 is caught on the step 2110 formed inside the hole, and the vibrating body fixing portion 4000 fixes the transducer cell 3000 while closely attaching the catching protrusion 3432 and the step 2110 by being screw-coupled into the hole.

In this case, a rubber ring 3434, which is a sealing member, is disposed in the groove 3433 formed on the catching protrusion of the metal vibrating body to prevent the fluid for ultrasound transmission from permeating into inside, and the transducer cell 3000 for generating ultrasound waves is independently fastened along a curved inner surface 2200 of the dome-shaped support 2000.

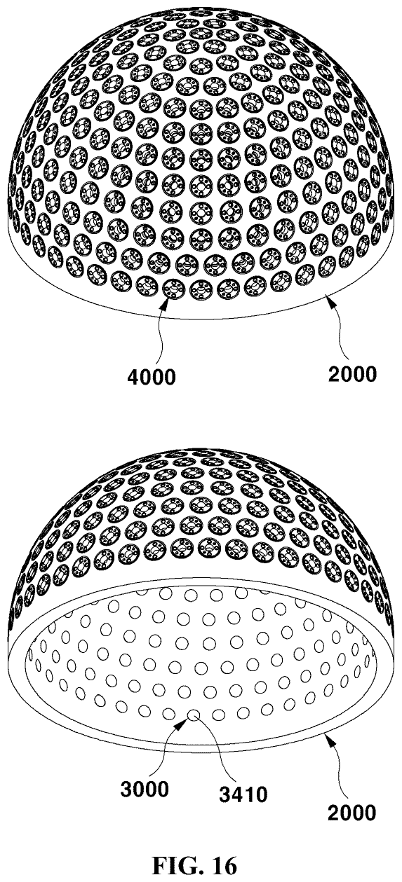

FIG. 16 shows the overall structure of a focused ultrasound transducer array completed according to an embodiment of the present invention. It shows the dome-shaped support 2000 and a state in which the ultrasound focusing transducer cells 3000 are inserted one by one into the plurality of holes formed on the support 2000 and are fixed by the vibrating body fixing portion 4000.

In this way, in the focused ultrasound transducer array according to the present invention, the ultrasound focusing transducer cells having a predetermined curvature are arranged so that the foci of the entire dome-shaped ultrasound focusing transducers can be easily controlled, and when some of the transducer cells for ultrasound focusing are damaged, only the damaged area can be simply disassembled and repaired. In addition, compared to the case where a planar ultrasound oscillator is attached to the existing dome-shaped support, scattering of ultrasound can be suppressed by installing an ultrasound focusing unit cell whose curvature is similar to the curvature of the dome-shaped support, thereby capable of accurately hitting the patient's treatment area. In addition, ultrasound energy can be effectively utilized in the transducer cell for ultrasound focusing by using the metal vibrating body that can amplify the displacement of piezoelectric ceramic on the ultrasound radiation surface. Through such a dome-shaped focused ultrasound transducer array, it can be applied to various medical ultrasound diagnostic devices or medical treatment devices.

1. A focused ultrasound transducer comprising:
a dome-shaped support and a plurality of ultrasound transducer cells coupled to the dome-shaped support,
wherein the ultrasound transducer cell includes a metal vibrating body whose first surface is disposed toward an inner side thereof, which is a concave direction of the dome-shaped support, and a piezoelectric ceramic element disposed on a second surface, which is a surface opposite to the first surface of the metal vibrating body, and
the first surface of the metal vibrating body has a predetermined radius of curvature so as to be concave toward the inner side of the dome-shaped support.

2. The focused ultrasound transducer of claim 1, wherein
the radius of curvature of the first surface of the metal vibrating body is the same as a radius of curvature of the inner side of the dome-shaped support.

3. The focused ultrasound transducer of claim 1, wherein
the radius of curvature of the first surface of the metal vibrating body is smaller than the radius of curvature of the inner side of the dome-shaped support.

4. The focused ultrasound transducer of claim 1, wherein
a seating groove is formed on the second surface of the metal vibrating body and the piezoelectric ceramic element is seated in the seating groove.

5. The focused ultrasound transducer of claim 1, wherein
a plurality of holes are formed in the dome-shaped support, and the plurality of ultrasound transducer cells are respectively inserted into the plurality of holes.

6. The focused ultrasound transducer of claim 5, wherein
an inner wall of the hole and an outer periphery of the metal vibrating body is screw-coupled to each other.

7. The focused ultrasound transducer of claim 5, wherein
the hole has a catching step inside thereof, and a catching portion that restricts an inward movement of the dome-shaped support by the catching step is formed on the metal vibrating body of the ultrasound transducer cell.

8. The focused ultrasound transducer of claim 7, wherein
a first wall extending from an edge of the second surface may be formed, and the catching portion may be formed on an outer peripheral part of the first wall.

9. The focused ultrasound transducer of claim 8, further comprising:
a vibrating body fixing portion that fixes the ultrasound transducer cell by inserting the transducer cell into the hole from an outside of the dome-shaped support to make the catching portion of the metal vibrating body and the catching step of the hole come into close contact with each other.

10. The focused ultrasound transducer of claim 9, wherein
the vibrating body fixing portion has a bolt structure that has a through-hole and is screw-coupled to the inner wall of the hole.

11. The focused ultrasound transducer of claim 7, wherein
a sealing member is disposed on a surface where the catching portion comes into contact with the catching step of the hole.

12. The focused ultrasound transducer of claim 1, wherein
the metal vibrating body is made of stainless steel.

13. The focused ultrasound transducer of claim 1, wherein
the dome-shaped support is made of an engineering plastic material.

14. A medical ultrasound device comprising the focused ultrasound transducer according to claim 1.
